(19) United States
(12) Patent Application Publication
(10) Pub. No.: US 2016/0121072 A1
(43) Pub. Date: May 5, 2016

(54) Obstructive Sleep Apnea Treatment Device

(71) Applicants: Andrew Cole Smith, Mico, TX (US); Ryan Anthony Brabant, San Antonio, TX (US); Jesse Allen Beavers, Boerne, TX (US); Joshua Anthony Lademora, Sayville, TX (US)

(72) Inventors: Andrew Cole Smith, Mico, TX (US); Ryan Anthony Brabant, San Antonio, TX (US); Jesse Allen Beavers, Boerne, TX (US); Joshua Anthony Lademora, Sayville, TX (US)

(21) Appl. No.: 14/932,907

(22) Filed: Nov. 4, 2015

Related U.S. Application Data
(60) Provisional application No. 62/074,852, filed on Nov. 4, 2014.

(51) Int. Cl. A61M 16/20; A61M 16/06

(52) U.S. Cl. CPC A61M 16/208; A61M 16/0616

(57) ABSTRACT

An obstructive sleep apnea treatment device is disclosed herein. In embodiments the device eliminates the need for large peripheral equipment such as an air pump and hoses commonly associated with current treatment devices. In embodiments, the device is comprised of a main mask body component, at least one expiratory or exhalation pressure valve, at least one inspiratory or inhalation valve. In other embodiments, the device is further comprised of a forehead cushion component, a facial seal component, and an attachment component. In embodiments, the main mask body component is a nasal covering mask configured with a positive end expiratory adjustable pressure valve to generate a resistance on a patient's exhalation thus pressuring the nasal airways without the need of an air pump or electrical power source. Positive pressurization of the airways reduces the occurrence of apneas.

17

### CROSS-REFERENCE TO RELATED APPLICATIONS

This application claims the benefit under Title 35 United States Code §119(e) of U.S. Provisional Patent Application Ser. No. 62/074,852; Filed: Nov. 4, 2014, the full disclosure of which is incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention relates generally to a device for the treatment of obstructive sleep apnea. More specifically, the present invention relates to a device for the treatment of obstructive sleep apnea through a mask that eliminates the need for large peripheral equipment such as an air pump and hoses commonly associated with current treatment devices.

### BACKGROUND OF THE INVENTION

Without limiting the scope of the disclosed device, the background is described in connection with a novel device used in the treatment of obstructive sleep apnea.

The field's prior art reflects many approaches and devices in providing a means for the treatment of obstructive sleep apnea.

Obstructive sleep apnea (OSA) is a sleep disorder that affects millions worldwide, and is characterized by shallow or infrequent breathing resulting from complete or partial blockage of a patient's airway multiple times during sleep as shown in FIG. 1. OSA causes many problems to a patient's sleep including oxygen deprivation of vital organs and irregular heart rhythms.

The main concern for sleep apnea patients is complete blockage of the airway causing breathing to cease for a lengthy period of time. Studies have shown that patients, unaware of their sleep apnea, stop breathing 10 to 15 times an hour for up to 30 seconds at a time [1]. Patients with sleep apnea can be fatigued and overweight, as their sleep is disrupted every night. Obese patients are more likely to present with sleep apnea as the airway gets blocked due to the excess weight on their necks. Other risk factors of obstructive sleep apnea include large tonsils, large tongue, small jawbone, family history of sleep apnea, and nasal obstruction. Snoring, a lesser side effect of sleep apnea, affects 40% of patients over the age of 40 [2]. Although not all patients who snore have sleep apnea, snoring is a partial blockage of the airway as shown in FIG. 2, and can lead to the complete blockage of the airway and sleep apnea.

When the airway is completely blocked, oxygen levels drop while carbon monoxide levels rise. This causes a spike in blood pressure and heart rate as the body forces itself to breathe. This causes an estimated 50-70% of patients with sleep apnea to develop hypertension (high blood pressure), thus increasing the likelihood of a heart attack, stroke, Coronary Heart Disease, and Chronic Heart Failure (in rare cases). Approximately 40-60% of patients who have stokes are later found to have obstructive sleep apnea, proving the severity of the condition [3].

FIG. 3 and FIG. 4 illustrates the treatment currently used for sleep apnea, a continuous positive airway pressure (CPAP). CPAP works by applying mild air pressure using an air pump fed to a mask that keeps the airway open. The power source and pump forcefully push air through tubes to a mask that maintains positive airway pressure throughout the patient's airway preventing its collapse. The apparatus consists of a mask fitted over the nose and/or mouth held in place by straps, a tube connects the mask to a pump, and a pump pressurizes air [4]. However, there are drawbacks for using a CPAP machine. For example, CPAP users may experience: difficulty tolerating forced air, feelings of claustrophobia, dry mouth, annoyance due to noise, the need for an expensive power source, the large and uncomfortable tubes that connect the mask to the air pump, the swallowing of air being pumped through the mask causing burping, and the constant air flow causing patients to feel like they are choking or suffocating [5].

An alternative to CPAP is expiratory positive airway pressure (EPAP), which works by applying a resistance on exhalation to maintain positive airway pressure preventing the collapse of the airway. As a patient exhales the resistance creates an increase in pressure in the airway making the pressure in the airway higher than atmospheric air (i.e. positive airway pressure) which forces the airway to remain open allowing for normal air intake. A study on sleep apnea patients using EPAP devices showed that OSA episodes were reduced by up to 90% while apnea durations were reduced by approximately 72% [6]. However, the current state of the art is a disposable device that does not allow the patient to adjust the pressure levels of their device.

In view of the foregoing, it is apparent that there exists a need in the art for a device to treat obstructive sleep apnea, which overcomes, mitigates, or solves the above problems in the art. It is the purpose of this invention to fulfill this and other needs in the art, which will become apparent to the skilled artisan once given the following disclosure.

### BRIEF SUMMARY OF THE INVENTION

The present invention, therefore, provides for a device for the treatment of obstructive sleep apnea through a mask that eliminates the need for large peripheral equipment such as an air pump and hoses commonly associated with current treatment devices. This is accomplished through the use of at least one one-way adjustable pressure exhalation valve and at least one one-way inhalation valve.

In embodiments the device eliminates the need for large peripheral equipment such as an air pump and hoses commonly associated with current treatment devices. In embodiments, the device is comprised of a main mask body component, at least one expiratory or exhalation pressure valve, at least one inspiratory or inhalation valve. In other embodiments, the device is further comprised of a forehead cushion component, a facial seal component, and an attachment component. In embodiments, the main mask body component is a nasal covering mask configured with a positive end expiratory adjustable pressure valve to generate a resistance on a patient's exhalation thus pressuring the nasal airways without the need of an air pump or electrical power source. Positive pressurization of the airways reduces the occurrence of apneas.

In embodiments, the exhalation adjustable pressure valve is configured with a spring to supply the correct pressure or resistance. Adjusting the spring in the positive end expiratory pressure valve yields a range of airway pressures, from 5 to 20 centimeters of water, to meet the physician prescribed pressure for the patient. In embodiments, the inhalation valves are dual independent flap valves that assist inhalation by offering low-resistance which correlates into low negative inhalation pressures. This design has few components and minimal moving parts ensuring a rapid rate and ease of production at minimal cost. Additionally, this can keep the overall weight below 3 ounces.

It is an object of the invention to provide a device for the treatment of obstructive sleep apnea through a mask without hoses and power sources. It is another object of the invention to provide an alternative to a CPAP mask that is an efficient and effective device for the treatment of obstructive sleep apnea that is light, compact, and easy to wear. It is a further object of the invention to provide a device for the treatment of obstructive sleep apnea through a mask, which allows for incremental, adjustable, or variable positive pressure settings that are easily adjustable by a physician or user of the mask.

In summary, the present invention discloses a device for the treatment of obstructive sleep apnea. More specifically, the present invention relates to a device for the treatment of obstructive sleep apnea through a mask that eliminates the need for large peripheral equipment such as an air pump and hoses commonly associated with current treatment devices.

### BRIEF DESCRIPTION OF THE SEVERAL VIEWS OF THE DRAWINGS

The accompanying drawings, which are incorporated in and form a part of the specification, illustrate a preferred embodiment of the present invention, and together with the description, serve to explain the principles of the invention. It is to be expressly understood that the drawings are for the purpose of illustration and description only and are not intended as a definition of the limits of the invention. In the drawings:

FIG. 1 is an internal side view of an individual illustrating a blocked airway;

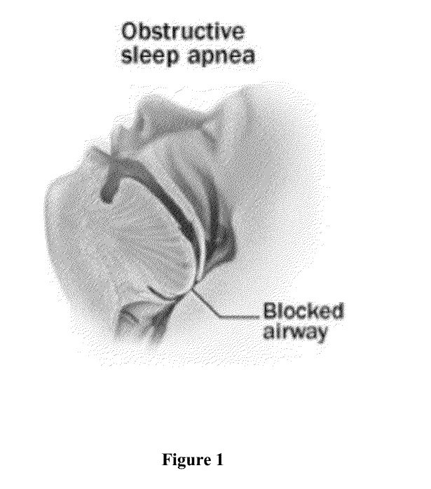

FIG. 2 is another internal side view of an individual illustrating a blocked airway caused by the closure by the soft palate and tongue;

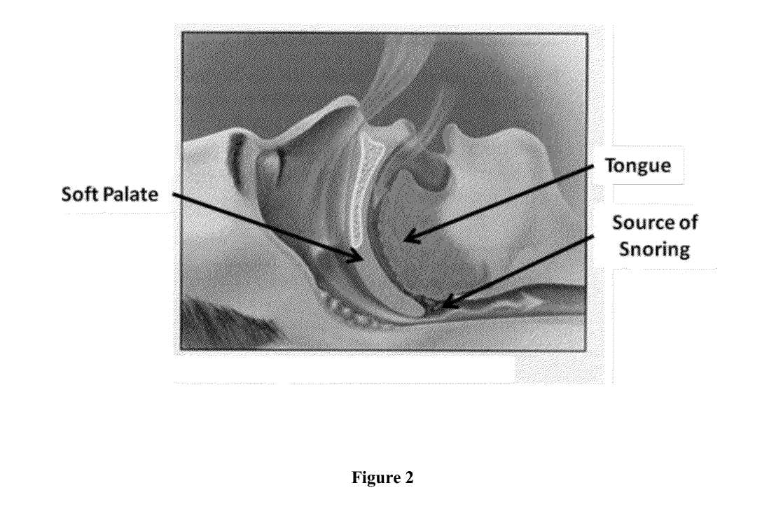

FIG. 3 is a front perspective view of an example of a current continuous positive airway pressure mask;

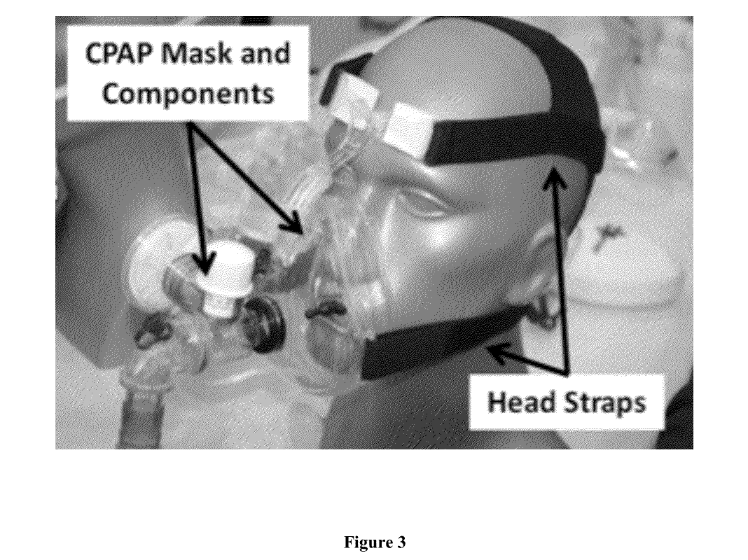

FIG. 4 is an illustration of the air pump and motor of an example of a current continuous positive airway pressure mask;

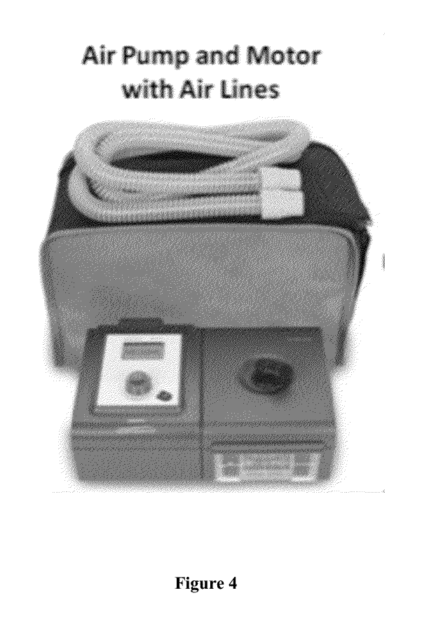

FIG. 5 is an environmental front perspective view of the obstructive sleep apnea treatment device in accordance with the teachings of the present disclosure;

FIG. 6 is a front perspective view showing the main body component, the exhalation pressure valve, the inhalation valve, and the forehead foam component of the obstructive sleep apnea treatment device in accordance with the teachings of the present disclosure;

FIG. 7 is an exploded front perspective view showing the main body component, the exhalation pressure valve, the inhalation valve, and the forehead foam component of the obstructive sleep apnea treatment device in accordance with the teachings of the present disclosure;

FIG. 8 is a front perspective view showing the exhalation pressure valve of the obstructive sleep apnea treatment device in accordance with the teachings of the present disclosure;

FIG. 9 is an exploded front perspective view showing the exhalation pressure valve of the obstructive sleep apnea treatment device in accordance with the teachings of the present disclosure;

FIG. 10 is a side sectional view of the obstructive sleep apnea treatment device illustrating the air flow through the inhalation valve during the inhalation process in accordance with the teachings of the present disclosure;

FIG. 11 is a side sectional view of the obstructive sleep apnea treatment device illustrating the air flow through the exhalation pressure valve during the exhalation process in accordance with the teachings of the present disclosure;

### DETAILED DESCRIPTION OF THE INVENTION

Disclosed herein is a device, system, and method of use for attracting and catching fish. The numerous innovative teachings of the present invention will be described with particular reference to several embodiments (by way of example, and not of limitation).

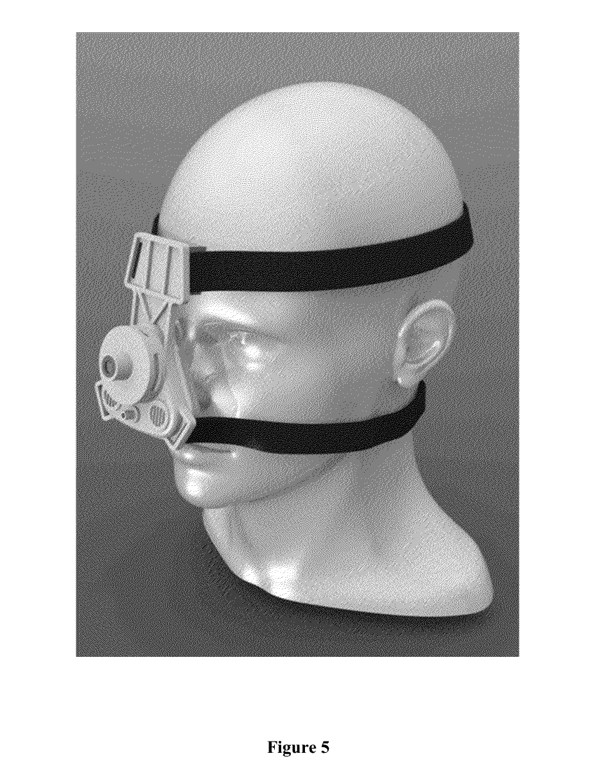

Reference is first made to FIG. 5, an environmental front perspective view of the obstructive sleep apnea treatment device in accordance with the teachings of the present disclosure. Illustrated here is an embodiment of the device being comprised of a main mask body component, a one-way expiratory or exhalation pressure valve, two one-way inspiratory or inhalation valves, a forehead cushion component, a facial seal component, and an attachment component. In this illustration the attachment components are two straps for attaching the main mask body component to the user of the device. In other embodiments, the attachment component may be for example and not a limitation, an adhesive or Velcro. This illustration provides a rendering on how the device in an embodiment will attach to the user. The main mask body component with the integrally formed or attached facial seal component forms an air tight seal between the device and the user of the device around the nasal region. That is only the nasal region is covered. In other embodiments, the air tight seal is formed between the device and the user of the device around the nasal and oral region. That is the nasal region as well as the oral region is covered.

The device allows for incremental positive-pressure settings easily adjustable by the physician or user using a gauged fixture on the obstructive sleep apnea treatment device. The device in an embodiment covers the nostrils and is small enough for easy travel. The device is wearable throughout an entire sleep period. The one-way exhalation adjustable positive pressure valve includes a one-way resistance applied on exhalation but not on inhalation. The one-way exhalation adjustable positive pressure valve is also removable and replaceable. The device is able to maintain the airway (pressure within the mask) at a selected pressure between 5 and 20 cm of H₂O. Meeting the pressure differential range of 5-20 cm of water (ΔP) is done by first producing the PEEP valve so that the internal spring and tapered poppet valve offer the necessary resistance force.

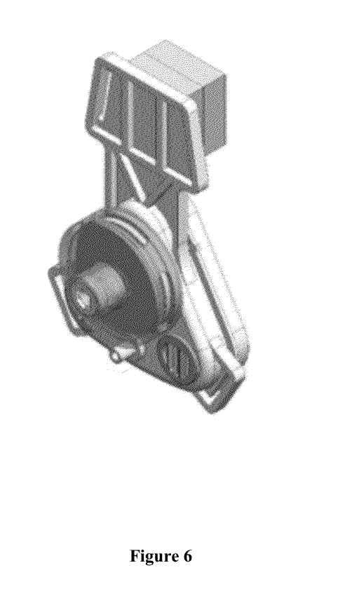

Reference is next made to FIG. 6, a front perspective view showing the main mask body component, the one-way exhalation pressure valve, the two one-way inhalation valves, and the forehead foam component of the obstructive sleep apnea treatment device in accordance with the teachings of the present disclosure. This illustration provides an assembled illustration of the base components of the device. In an embodiment, the device is an adjustable pressure EPAP nasal mask. The device generates a positive airway pressure inside the nasal passage on inhalation causing the nasal walls to be forced into an open position. This alleviates the condition responsible for OSA. The device incorporates an adjustable positive end expiratory pressure (PEEP) valve also referred to as the one-way exhalation adjustable pressure valve for regulating the exhalation pressure. The valve is a removable component of the mask.

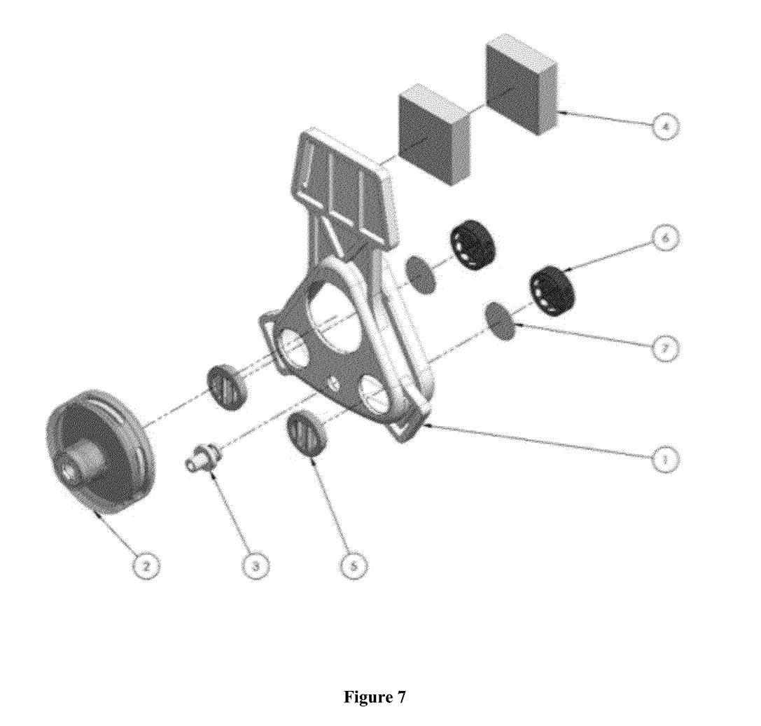

Reference is now made to FIG. 7, an exploded front perspective view showing the main mask body component, the exhalation pressure valve, the inhalation valve, and the forehead foam component of the obstructive sleep apnea treatment device in accordance with the teachings of the present disclosure. Illustrated here are the base components of the device. Here shown is the main mask body component 1, the one-way exhalation adjustable pressure valve 2, tubing barb 3, the forehead cushion components 4, and two one-way inhalation valves 5, 6, 7. In other embodiments, the device is further comprised of a seal component. In yet another embodiment, the device is also comprised of an attachment component. The forehead cushion components 4 in an embodiment are soft foam squares attached to the main mask body component 1. The main mask body component 1 functions as the base whereby the other components are attached to form the complete EPAP mask. The mask is configured to receive and have attached to it the other components. In an embodiment, the one-way exhalation adjustable pressure valve 2 is attached to the central front of the main mask body component 1. The one-way inhalation valves 5, 6, 7 are comprised in an embodiment of the front side housing cap with airway ports 5, the flap valve or flap disk 6, and the back side housing cap with inspiration ports 6. The flap valve 6 on exhalation moves forward and covers the airway ports not allowing air to leave from the air entering the inspiration ports. On inhalation, the flap valve 6 moves to the back of the back side housing cap with inspiration ports 6 allowing the air entering the inspiration ports to exit through the airway ports of the front side housing cap 5.

By leaving the mouth uncovered this device or EPAP mask prevents the possibility of suffocation. The mask is also designed so that it has a “forced produced” seal between the patient's face and the mask's silicone or silicone-like material seal component, thus allowing generation of a positive pressure differential between the ambient room pressure and this inside of the mask.

The sealing force is achieved with an adjustable length elastic head strap (attachment component) that is attached to the mask and will be positioned behind the patient's head. For extra comfort a foam backed tab feature (forehead cushion component) will be located on the upper portion of the mask just above the nose-bridge, and will rest on the patient's forehead. This will redistribute the pressure of the mask on the nose bridge onto the forehead.

The pressure differential between the internal side of the mask and the ambient room pressure will be obtained by utilizing a soft contour conforming material (seal component) as the contact area between the mask and patient's face. Thus, as exhalation occurs the exiting air is prevented from passing through the PEEP valve until the restrictive spring force of the internal PEEP valve spring is overcome by natural breathing. The valve spring can be compressed to various lengths by rotating the adjustment knob. The knob is attached to the PEEP valve body via thread engagement, thus rotating it one direction will increase the amount of compression on the spring and increase the functional pressure differential. Rotating the opposite direction will decrease the compression and pressure differential.

Inhalation pressure differentials will be minimized with the use of mask ventilation ports or one-way inhalation valves covered with soft membrane flaps attached to the inside of the mask. There will be two ventilation ports, one on each side of the mask and each with a separate inhalation flap. The membrane will consist of a thin silicone or silicone-like material. In its normal resting state and during exhalation, the inhalation flaps will rest against the ventilation ports, effectively creating a seal. On inhalation the flaps will offer negligible resistance and allow for adequate flow rates.

While creating a resistance on exhalation creates a positive pressure that keeps the airway open, a resistance on inhalation causes a negative pressure that would force the airway to close. With this in mind, the inlet ports were designed to have a minimal resistance to not restrict inhalation.

The device does not require a power supply and/or pump, as with more standard CPAP devices, and due to this can operate in many more environments and be much quieter. The device is able to provide a range of positive pressure settings in a single EPAP device, which is currently not provided for in the prior art. As a design consisting of minimal number of components this device can be easily cleaned with soap and water before being reused.

In other embodiments, the device full-size EPAP mask that covers the nose and mouth.

The tubing barb 3, is an attachment point where sensors and other equipment may be attached for monitoring and managing the pressure within the device.

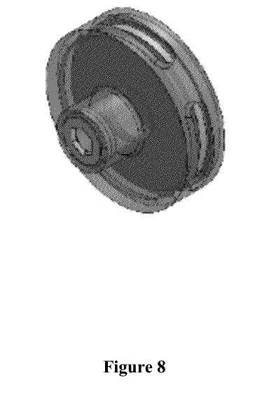

Reference is next made to FIG. 8, a front perspective view showing the exhalation pressure valve also referred to as a one-way exhalation adjustable pressure valve of the obstructive sleep apnea treatment device in accordance with the teachings of the present disclosure.

The device is characterized as an adjustable pressure EPAP nasal mask capable of treating OSA by generating a positive airway pressure inside the nasal passages upon exhalation, causing the nasal passages to be forced open. This is accomplished by incorporating an adjustable PEEP valve (one-way exhalation adjustable pressure valve) into the mask that regulates the exhalation pressure. The PEEP valve is designed as tapered poppet valve.

To minimize physiological effects by maintaining a low negative pressure on inhalation, the inhalation valves are designed as low resistant flap valves.

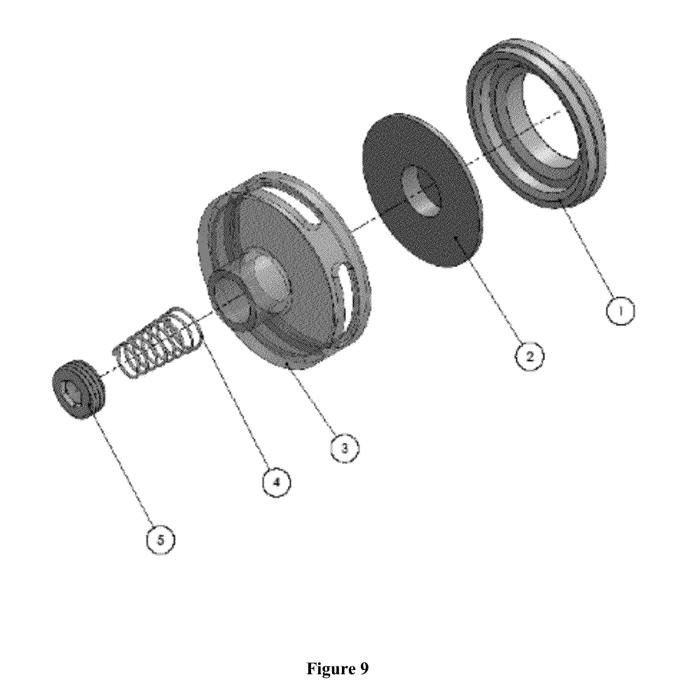

Reference is now made to FIG. 9, an exploded front perspective view showing the exhalation pressure valve of the obstructive sleep apnea treatment device in accordance with the teachings of the present disclosure.

In embodiments, the exhalation pressure valve of the obstructive sleep apnea treatment device is comprised of a back side housing cap 1, a flap valve or flap disk 2, a front side housing cap 3 with expiration ports, the exhalation pressure valve spring 4, and the adjustment knob 5.

The adjustment knob 5 is attached to the exhalation valve spring 4. The exhalation pressure valve spring 4 passes through the front side housing cap 3 and abuts or is attached to the center receiving cavity of the flap valve or flap disk 2. The flap valve or flap disk 2 is positioned internal and between the fronts side housing cap 3 and the back side housing cap 1. On inhalation, the flap valve or flap disk 2 moves to the back and presses against the back side housing cap 1. This closes off the exhalation pressure valve and does not allow air to enter into the mask from the exhalation ports on the front side housing cap 3. On exhalation, the flap valve or flap disk 2 moves to the front and abuts or presses against the internal front of the front side housing cap 3. This opens up the exhalation pressure valve and allows air to exit the mask through the exhalation ports on the front side housing cap 3.

In embodiments, the adjustment knob is threaded into the extruded left most cavity portion of the front side housing cap 3. As the adjustment knob is threaded out or to the left of the front side housing cap 3, the resistive force of the spring applied to the flap valve or flap disk 2 is reduced. As the adjustment knob is threaded in or to the right inside of the front side housing cap 3, the resistive force of the spring applied to the flap valve or flap disk 2 is increased.

In embodiments, a circular PEEP valve piston with a diameter of 1.25″, the required resistive force provided by the spring was determined. The necessary resistive forces of 0.0873 and 0.3491 lbf are determined for the minimum and maximum target differential pressure of 5 and 20 cm of water, respectively. These values give a spring constant of 1.3 lbf/in. to meet the force requirements.

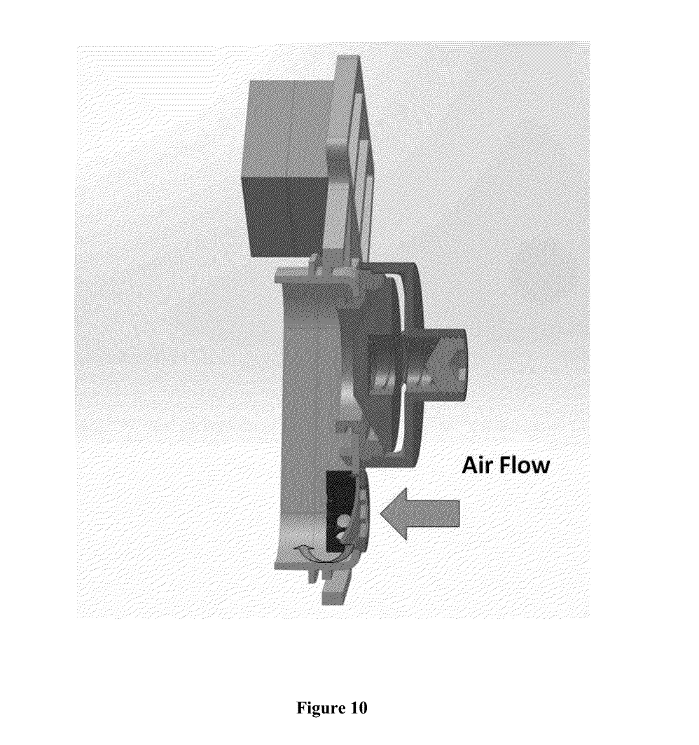

Reference is next made to FIG. 10, a side sectional view of the obstructive sleep apnea treatment device illustrating the air flow through the inhalation valve during the inhalation process in accordance with the teachings of the present disclosure. Illustrated in this figure is the inhalation process whereby the air flow is directed through the two one-way inhalation valves. Air cannot enter through any other means into the mask.

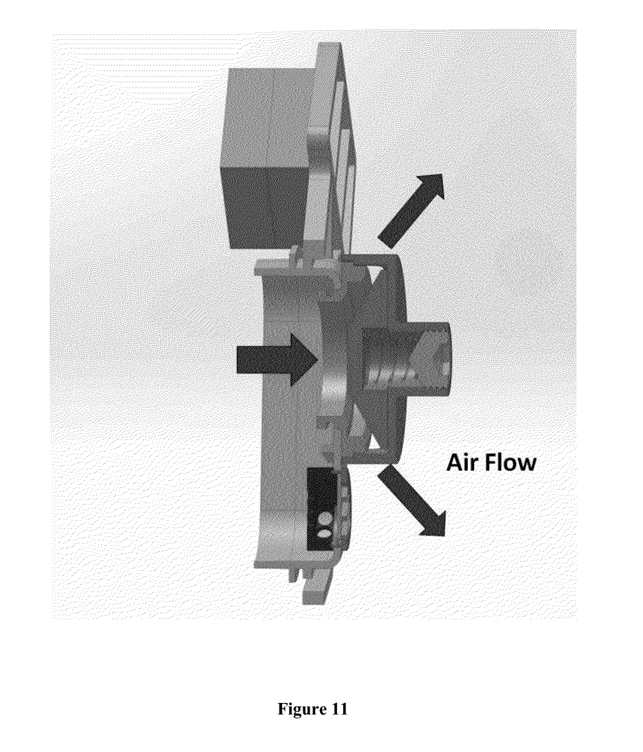

Reference is lastly made to FIG. 11, a side sectional view of the obstructive sleep apnea treatment device illustrating the air flow through the exhalation pressure valve during the exhalation process in accordance with the teachings of the present disclosure. Illustrated in this figure is the exhalation process whereby the air flow is directed out through the one one-way adjustable pressure exhalation valve. Air cannot exit through any other means out of the mask.

In brief, the present invention relates to a device for the treatment of obstructive sleep apnea through a mask which eliminates the need for large peripheral equipment such as an air pump and hoses commonly associated with current treatment devices. This is accomplished through the use of at least one one-way exhalation adjustable pressure valve and at least one one-way inhalation valve.

The disclosed device is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention. Terms such as “a”, “an”, and “the” are not intended to refer to only a singular entity, but include the general class of which a specific example may be used for illustration. The terminology herein is used to describe specific embodiments of the invention, but their usage does not delimit the disclosed device, system, or method, except as may be outlined in the claims.

Any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device, system, and method of use described herein. Such equivalents are considered to be within the scope of this invention and are covered by the claims.

All publications, references, patents, and patent applications mentioned in the specification are indicative of the level of those skilled in the art to which this invention pertains. All publications, references, patents, and patent application are herein incorporated by reference to the same extent as if each individual publication, reference, patent, or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device disclosed and claimed herein can be made and executed without undue experimentation in light of the present disclosure. While the device of this invention has been described in terms of preferred embodiments, it will be apparent to those skilled in the art that variations may be applied to the device without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

### REFERENCES

1. “Sleep Apnea” Catching the Culprit: Sleep Apnea. N.p., 2011. Web. 21 Feb 2014.
   http://blog.lib.umn.edu/hamdi002/blog/2011/10/catching-the-culprit-sleep-apnea.html
2. “Statistic Brain” Snoring Statistics. N.p. 2013. Web. 21. Feb. 2014.
   http://www.statisticbrain.com/snoring-statistics/
3. “About.com”. Why Sleep Apnea May Be Deadly. N.p. 2014. Web 21 Feb. 2014.
   http://sleepdisorders.about.com/od/sleepandgeneralhealth/a/deadlyapnea.htm
4. “What Is CPAP?”-NHLBI, NIH. N.p., n.d. Web. 16 Feb. 2014.
   https://www.nhlbi.nih.gov/health/health-topics/topics/cpap/.
5. “Diseases and Conditions.” CPAP Machines: Tips for Avoiding 10 Common Problems. N.p., n.d. Web. 16 Feb. 2014. http://www.mayoclinic.org/diseases-conditions/sleep-apnea/in-depth/cpap/art-20044164?pg=2.
6. Mahadevia, Akshay K., Ergun Onal, and Melvin Lopata. “Effects of Experatory Positive Airway Pressure on Sleep-Induced Abnormalities in Patients with Hypersomnia-Sleep Apnea Syndrome.” American Review of Respiratory Disease 128 (1983): 708-11.
7. “Provent Starter Kit.” Starter Kit. N.p., n.d. Web. 16 Feb. 2014.
   http://www.proventtherapy.com/starter-kit.php.
8. “EPAP: An Effective CPAP Alternative?” Pulmonary Reviews. N.p., 2010. Web. 16 Feb. 2014.
   http://www.pulmonaryreviews.com/Article.aspx?ArticleId=7DlmLkl7/dI=&FullText=1
9.-Century TA-2198 Material Properties: <http://www.centuryspring.com/pdfs/377-381APPENDIX-A.pdf>
10. Typical Weight of a Human Head: http://en.wikipedia.org/wiki/Human_head
11. Respiratory Minute Volume:
   http://rtboardreview.com/public/tables_lists/minute_{—} alveolarventilation.htm
12. J. A. Beavers, R. Brabant, J. Lademora, and A. Smith. N.p., 2014 “OSATD Drawing Package.” Unpublished manuscript, METech, San Antonio, Tex., United States of America.
13. J. A. Beavers, R. Brabant, J. Lademora, and A. Smith. N.p., 2014 “Test Plan: Obstructive Sleep Apnea Treatment Device (OSATD).” Unpublished manuscript, METech, San Antonio, Tex., United States of America.

What is claimed is:

1. A device for treating obstructive sleep apnea comprising:
a main mask body component having a facial seal component;
at least one one-way expiratory or exhalation pressure valve;
at least one one-way inspiratory or inhalation valve;
and an attachment component.

2. The device of claim 1, further comprising a forehead cushion component.

3. The device of claim 1, wherein said main mask body component is configured to seal only the nasal region.

4. The device of claim 1, wherein said main mask body component is configured to seal only the nasal and oral region.

5. The device of claim 1, wherein said device is configured with only one one-way expiratory or exhalation pressure valve.

6. The device of claim 1, wherein said device is configured with two one-way inspiratory or inhalation valves.

7. The device of claim 1, wherein said device is configured with only one one-way expiratory or exhalation pressure valve; two one-way inspiratory or inhalation valves; and wherein said main mask body component seals the nasal region only.

8. The device of claim 1, wherein said one-way inspiratory or inhalation valve is comprised of a front side housing cap with airway ports;
a flap valve or flap disk; and
a back side housing cap with inspiration ports.

9. The device of claim 1, wherein said one-way expiratory or exhalation pressure valve is comprised of
a back side housing cap;
a flap valve or flap disk;
a front side housing cap with expiration ports;
an exhalation pressure valve spring; and
an adjustment knob.

10. The device of claim 8, wherein said one-way expiratory or exhalation pressure valve is comprised of
a back side housing cap;
a flap valve or flap disk;
a front side housing cap with expiration ports;
an exhalation pressure valve spring; and
an adjustment knob.

11. The device of claim 1, wherein said one-way expiratory or exhalation pressure valve is configured to provide a pressure range of about 5 to 20 cm of H₂O.

12. The device of claim 9, wherein said one-way expiratory or exhalation pressure valve is configured to provide a pressure range of about 5 to 20 cm of H₂O.

13. The device of claim 7, wherein said one-way inspiratory or inhalation valve is comprised of a front side housing cap with airway ports, a flap valve or flap disk, and a back side housing cap with inspiration ports; and wherein said one-way expiratory or exhalation pressure valve is comprised of a back side housing cap, a flap valve or flap disk, a front side housing cap with expiration ports, an exhalation pressure valve spring, and an adjustment knob.

14. The device of claim 9, wherein said adjustment knob is configured to adjust the exhalation pressure valve spring to provide a pressure range of about 5 to 20 cm of H₂O.

15. The device of claim 10, wherein said adjustment knob is configured to adjust the exhalation pressure valve spring to provide a pressure range of about 5 to 20 cm of H₂O.

16. The device of claim 9, wherein said adjustment knob is configured to be threaded into said front side housing cap to adjust the exhalation pressure valve spring to provide a pressure range of about 5 to 20 cm of H₂O.

17. The device of claim 10, wherein said adjustment knob is configured to be threaded into said front side housing cap to adjust the exhalation pressure valve spring to provide a pressure range of about 5 to 20 cm of H₂O; and wherein said main mask body component is configured to seal only the nasal region.

18. A device for treating obstructive sleep apnea comprising:
a main mask body component having a facial seal component; and at least one one-way expiratory or exhalation pressure valve;

19. The device of claim 18, wherein said one-way expiratory or exhalation pressure valve is comprised of
a back side housing cap;
a flap valve or flap disk;
a front side housing cap with expiration ports;
an exhalation pressure valve spring; and
an adjustment knob.

20. The device of claim 19, wherein said main mask body component is configured to seal only the nasal region; and wherein said device is configured with only one one-way expiratory or exhalation pressure valve; and wherein said one-way expiratory or exhalation pressure valve is configured to provide a pressure range of about 5 to 20 cm of H₂O
